(19) United States
(12) Patent Application Publication
(10) Pub. No.: US 2020/0172980 A1
(43) Pub. Date: Jun. 4, 2020

(54) PROSTATIC CANCER MARKER, PCDH9, AND APPLICATION THEREOF

(71) Applicant: SHANGHAI CHANGHAI HOSPITAL, Shanghai (CN)

(72) Inventors: Yinghao SUN, Shanghai (CN); Shancheng REN, Shanghai (CN); Xiaolei SHI, Shanghai (CN); Yasheng ZHU, Shanghai (CN); Yue YANG, Shanghai (CN)

(73) Assignee: SHANGHAI CHANGHAI HOSPITAL, Shanghai (CN)

(21) Appl. No.: 16/481,311

(22) PCT Filed: Dec. 15, 2017
(86) PCT No.: PCT/CN2017/116359

§ 371 Date: Nov. 19, 2019

(30) Foreign Application Priority Data
Jan. 26, 2017 (CN) 201710057306.4

(51) Int. Cl. C12Q 1/6886

(52) U.S. Cl. CPC C12Q 1/6886; C12Q 2600/158

(57) ABSTRACT

The present invention provides a biomarker PCDH9 for molecular classification of prostate cancer and for evaluation of prostate cancer prognosis and uses thereof. The biomarker PCDH9 can be DNA or mRNA of PCDH9 or a protein encoded thereby. The present invention also provides a prostate cancer in vitro diagnostic product comprising a detection reagent of the biomarker PCDH9.

### REFERENCE TO SEQUENCE LISTING SUBMITTED VIA EFS-WEB

This application includes an electronically submitted sequence listing in .txt format. The .txt file contains a sequence listing entitled “2019-11-19_SequenceListingasFiled_6075-0119PUS1.txt” created on Nov. 4, 2019 and is 19,315 bytes in size. The sequence listing contained in this .txt file is part of the specification and is hereby incorporated by reference herein in its entirety

### TECHNICAL FIELD

The present invention relates to evaluation of prostate cancer prognosis, particularly to uses of PCDH9 as a biomarker for molecular classification of prostate cancer as well as for in evaluation of prostate cancer prognosis, and a kit thereof.

### BACKGROUND TECHNIQUE

Prostate Cancer (PCa) is known as a type of malignant tumor that poses threat to men. The incidence and mortality rates are the highest for men aged 70, which put prostate cancer a second place on the ranking of global cancer incidence rates and a sixth place for mortality rates. In China, the incidence rate of prostate cancer has been on the rise for recent years. The high incidence rate earns prostate cancer a place among the top 10 cancers in the medically and economically developed cities such as Beijing, Shanghai and Guangzhou. Epidemiology statistics have shown an increase in the incidence rate of prostate cancer in men from 1.71/100,000 to 7.9/100,000, approximately a growth of 13% over 1993 to 2005. It is estimated that the incidence rate will be exceeding 40/100,000 males by 2020, coming close to that in the western countries, and posing a major life-threatening risk to men. China, witnessing the aging of its society and the progress made in healthcare in general, finds the urging need for development in the research work of prevention and treatment of prostate cancer. Many patients will not be aware of their illness until it develops into a mid- or late-stage due to the fact that prostate cancer usually does not present any symptoms, with as many as 46.3% of patients have the cancer already partially advanced or spread to other parts of the body at their initial diagnosis. In cases where the cancer is spread, it is often time accompanied with poor prognosis and reduced quality of life. According to the information during 2004 to 2010 from SSER (Surveillance Epidemiology and End Results) database, the 5-year survival rate of localized prostate cancer remains 100% while only 28% for metastatic prostate cancer. In China, there can be seen blanks in the field of prostate cancer related researches, including its high heterogeneity, that efficient diagnosis and treatment of prostate cancer are faced with obstacles. Evaluation of prostate cancer prognosis also varies dramatically among individuals, from more than 10 years to only 2-3 years. Some clinical indicators, such as the Gleason Score, have been referred to for evaluating the malignancy grade of the cancer. These are still far from being accurate, as great inconsistency in prognosis evaluation has been found for patients with the same Gleason score. It is therefore of significance to find a biomarker for molecular classification of prostate cancer and its prognosis evaluation.

### SUMMARY OF THE INVENTION

According to a first aspect of the invention, there is provided a use of PCDH9 as a biomarker for molecular classification of prostate cancer as well as for in evaluation of prostate cancer prognosis, in which the diagnosis and prediction of prostate cancer are improved in terms of accuracy and specificity by means of detection of PCDH9.

PCDH9 (Protocadherin 9), as a member of the protocadherin family, plays a vital role in cell adhesion, nervous projection and synapse formation. In the present invention, a prostate cancer biomarker is referred to as a PCDH9 DNA, an mRNA or a protein encoded thereby. The DNA sequence of PCDH9 is:https://genome-asia.ucsc.edu/cgi-bin/hgc?hgsid=471531278_ygPZLWdraI1aa0SUyaxbvo6AikMn &g=htcGetDna2&table=&i=mixed&1=66302833&r=67230336&getDnaPos=chr13%3A66%2C302% 2C834-67%2C230%2C336&db=hg38&hgSeq.cdsExon=1&hgSeq.padding5=0&hgSeq.padding3=0&hgSeq.casing=upper&boolshad.hgSeq.maskRepeats=0&hgSeq.repMasking=lower&boolshad.hgS eq.revComp=0&submit=get+DNA. The mRNA sequence of PCDH9(https://www.ncbi.nlm.nih.gov/nuccore/NM_203487.2) is shown as SEQUENCE ID No. 1. The protein sequence encoded by the PCDH9 gene is shown as sequence ID No. 2. In the present invention, molecular classification of prostate cancer and evaluation of prostate cancer prognosis are achieved by means of determining the change in gene expression levels by determining whether is present any deletion or change in copy number of the PCDH9 gene or change in the expression level of mRNA or the protein encoded thereby.

In the present invention, the prostate cancer biomarker includes such a PCDH9 DNA whose nucleotide sequence having homology of at least 80%, 85%, 90%, 95%, or 99% to the sequence of said DNA PCDH9.

In the present invention, the prostate cancer biomarker includes such a PCDH9 mRNA whose nucleotide sequence having homology of at least 80%, 85%, 90%, 95%, or 99% to the sequence of said mRNA PCDH9 (SEQUENCE ID No. 1).

In the present invention, the prostate cancer biomarker includes such a protein whose amino acid sequence having homology of at least 80%, 85%, 90%, 95%, or 99% to the sequence of said PCDH9 protein (SEQUENCE ID No. 2).

The present invention also provides the use of reagent for detection of PCDH9 DNA as a product for molecular classification of prostate cancer. Said reagent includes but not limited to a specific nucleic acid probe for detecting a PCDH9 DNA. Molecular classification of prostate cancer is achieved by determining and quantifying whether is present any deletion or change in copy number of the PCDH9 gene.

The present invention also provides the use of reagent for detection of PCDH9 mRNA as a product for molecular classification of prostate cancer. Said reagent includes but not limited to a specific nucleic acid probe for detecting a PCDH9 mRNA. Molecular classification of prostate cancer is achieved by determining and quantifying whether is present any change in the expression level of PCDH9 mRNA. The expression level of the mRNA can be determined by the following methods: microarray technology, Northern blotting and quantitative PCR; the quantitative PCR is real-time quantitative PCR or multiplex PCR, etc.

The present invention also provides the use of reagent for detection of protein encoded by the PCDH9 gene as a product for molecular classification of prostate cancer. Said reagent includes but not limited to a specific antibody for detecting a protein encoded by the PCDH9 gene. Molecular classification of prostate cancer is achieved by determining and quantifying whether is present any change in the expression level of protein encoded by the PCDH9 gene. The expression level of protein encoded by the PCDH9 gene can be determined by known measures as immunohistochemistry, Western blotting, ELISA, RIA and mass spectrometry, etc.

The present invention also provides an in vitro diagnostic product for prostate cancer. Said in vitro diagnostic product comprises a reagent for specific detection of PCDH9 DNA, and/or a reagent for specific detection of PCDH9 mRNA, and/or a reagent for specific detection of protein encoded by the PCDH9 gene. Said in vitro diagnostic product can be used in molecular classification of prostate cancer as well as in evaluation of prostate cancer prognosis. Said reagent for specific detection of PCDH9 DNA includes but not limited to a nucleic acid probe, which specifically identifies the PCDH9 DNA. Said reagent for specific detection of PCDH9 mRNA includes but not limited to a nucleic acid probe, which specifically identifies the PCDH9 mRNA. Said reagent for specific detection of protein encoded by the PCDH9 gene includes but not limited to an antibody, which specifically identifies the protein encoded by the PCDH9 gene.

In the present invention, said in vitro diagnostic product for prostate cancer comprises a kit, a gene chip and a solid support, etc. Said solid support includes arrays, microarrays and protein arrays, etc.

The present invention also provides the use of a PCDH9 DNA, an mRNA or a protein encoded thereby in the preparation of pharmaceuticals for suppressing the proliferation, metastasis and invasion of prostate cancer cells.

The present invention also provides the use of a PCDH9 DNA, an mRNA or a protein encoded thereby in the preparation of pharmaceuticals for suppressing the expression of prostate cancer oncogenes, wherein said prostate cancer oncogenes include HOXB13 and ETS1, etc.

The present invention also provides the use of a PCDH9 DNA, an mRNA or a protein encoded thereby in the preparation of pharmaceuticals for suppressing the expression of marker of prostate cancer stem cells, wherein said marker of prostate cancer stem cells is ALDH1A1.

The present invention also provides the use of a PCDH9 DNA, an mRNA or a protein encoded thereby in the preparation of pharmaceuticals for promoting the expression of transfer inhibitors of prostate cancer cells, wherein said markers of prostate cancer stem cells metastasis inhibitors of prostate cancer cells include FOXOA and FOXP1, etc.

The present invention also provides the use of a PCDH9 DNA, an mRNA or a protein encoded thereby in the preparation of pharmaceuticals for promoting the expression of epithelial-mesenchymal transition marker, wherein said epithelial-mesenchymal transition marker is CDH1.

The present invention also provides a method for molecular classification of prostate cancer and evaluation of prostate cancer prognosis in patients who are diagnosed with prostate cancer, comprising the following steps:

a) detecting the DNA copy number of the PCDH9 gene, or the expression levels of mRNA or the protein encoded thereby in pathological samples of prostate cancer;

b) dividing the patients into a low expression group and a regular expression group by referring to the result of the previous step, wherein the low expression group is comprised of such patients in whom are seen deletion or decrease in the DNA copy number of the PCDH9 gene, or decrease in the expression levels of PCDH9 mRNA or the protein encoded thereby, compared to their control counterparts, and who are given a bad prognosis indicative of deteriorating and highly invasive tumors, poor prognosis for survival, likelihood of biochemical recurrence, distant metastasis or disease progression or death; whereas the regular expression group is presented with a result of the previous step reflecting the opposite.

The present invention is advantageous in that the novel biomarker PCDH9 for the diagnosis and prediction of prostate cancer can be used for molecular classification of prostate cancer as well as for in evaluation of prostate cancer prognosis with high specificity and high sensitivity. The in vitro diagnostic product for prostate cancer provided in the present invention which comprises a detection reagent of the PCDH9 biomarker, is convenient to use and demonstrates high accuracy, specificity and sensitivity.

### BRIEF DESCRIPTION OF THE DRAWINGS

FIG. 1 shows the correlation between the presence of deletion or expression level of PCDH9 and the degree of malignancy of prostate cancer. The tumor suppressor gene cluster PCDH9-DACH1-KLF5-LECT1-OLFM4 is localized in the segment of 13q21.31-q21.33 with a distinctive difference in copy number variation.

FIG. 2 shows the sequencing results of 65 samples respectively for prostate cancer and adjacent tissues. Significantly lower expression of PCDH9 is seen in the prostate cancer tissues in comparison to the adjacent tissues, which is observable in the prostate cancer tissues both with deletion mutation and in wild type of PCDH9.

FIG. 3 shows that significantly lower expression of PCDH9 is seen in the prostate cancer tissues with deletion mutation in comparison to the prostate cancer tissues of wild type, whereas a represents TCGA (The Cancer Genome Atlas) database and b represents the data published in Talyor 2010.

FIG. 4 shows that the expression level of PCDH9 in prostate cancer tissues is significantly decreased compared with that in the normal adjacent tissues, whereas a represents the GSE62872 database and b represents TCGA database.

FIG. 5 shows that the expression level of PCDH9 in prostate cancer tissues is higher compared with that in the metastatic prostate cancer tissues, the expression level of PCDH9 mRNA in normal tissues is significantly higher compared with tumor tissues and also higher than that in metastatic prostate cancer tissues, whereas a represents the GSE6811 database, b is the GSE21032 public database and c is the GSE35988 public database.

FIG. 6 shows that the expression level of PCDH9 in prostate cancer tissues in a high clinical stage is significantly lower compared with that in a low clinical stage (Glinsky 2004).

FIG. 7 shows that the expression level of PCDH9 mRNA in patients gradually goes down with the increase of PSA level (4-10, 10-20, >20) (Talyor 2010).

FIG. 8 shows the survival curves indicative of biochemical recurrence risk for populations with prostate cancer respectively with PCDH9 high expression and low expression, whereas a represents Glinsky 2004 and b TCGA database.

FIG. 9 shows the survival curves indicative of biochemical recurrence risk for populations with prostate cancer respectively with deletion in the DNA copy number of the PCDH9 gene or with normal DNA copy number, whereas a represents Memorial Sloan Kettering Cancer Center (MSKCC) database and b is TCGA database.

FIG. 10 shows, according to the statistics from Taylor 2010, deletion in the DNA copy number of the PCDH9 gene in the samples of metastatic prostate cancer tissues is significantly higher compared with of in situ in human prostate cancer.

FIG. 11 shows, according to the statistics from MSKCC database, deletion in the DNA copy number of the PCDH9 gene is significantly relevant with the reduction in survival period of patients with metastatic prostate cancer.

FIG. 12 shows, according to the statistics from Grasso 2010, deletion in the DNA copy number of the PCDH9 gene is significantly relevant with the reduction in survival period of patients with metastatic prostate cancer.

FIG. 13 shows the effects of PCDH9 as a tumor suppressor gene against the prostate cancer being proved by the in vitro experiments. The proliferative capacity, migration and invasion ability of the tumor cells were diminished after performing exogenous overexpression of PCDH9 (pReceiver is the control plasmid and PCDH9 is the plasmid with over expression), whereas a and d represent the proliferative capacity, b and e the invasion ability and c and f the migration ability.

FIG. 14 shows the effects of PCDH9 as a tumor suppressor gene against the prostate cancer being proved by the in vivo experiments. Whereas a and b represent the nude mouse models bearing subcutaneous tumors of prostate cancer cells DU145-PCDH9 with over expressed PCDH9, showing both the size and weight of the prostate cancer tumors are significantly reduced in comparison with the normal control. C as Immunohistochemistry (IHC) shows that, in the prostate cancer cells DU145-PCDH9 with over expressed PCDH9, the expression level of PCDH9 is significantly up-regulated and that of the cell proliferation marker Ki-67 significantly down-regulated.

FIG. 15 shows, according to the data analysis of gene expression profiling chip, the up-regulation of the expression of PCDH9 leads to the down-regulation of prostate cancer oncogenes (such as HOXB13 and ETS1), and cancer stem cell marker ALDH1A1, but up-regulation of transfer inhibitors (such as FOXOA and FOXP1) and epithelial-mesenchymal transition marker (such as CDH1).

FIG. 16 shows, the change in expression level of HOXB13, ETS1, FOXOA, FOXP1 and CDH1 by using real-time quantitative PCR method to confirm the up-regulation of the expression of PCDH9.

### DETAILED DESCRIPTION OF THE INVENTION

In the following description numerous specific details are set forth to provide a thorough understanding of the present invention. However, it will be apparent to those skilled in the art that the present invention may be practiced without these specific details. In other instances, well known elements, such as transformers and logic gates, have not been described in detail in order to not unnecessarily obscure the present invention.

### EXAMPLE 1

### The Expression of pcdh9 mRNA in the Prostate Cancer Tissues and the Normal Adjacent Tissues from Patients with Prostate Cancer

(1.1) Significant difference was observed in the expression level of PCDH9 mRNA in the prostate cancer tissues and the normal adjacent tissues

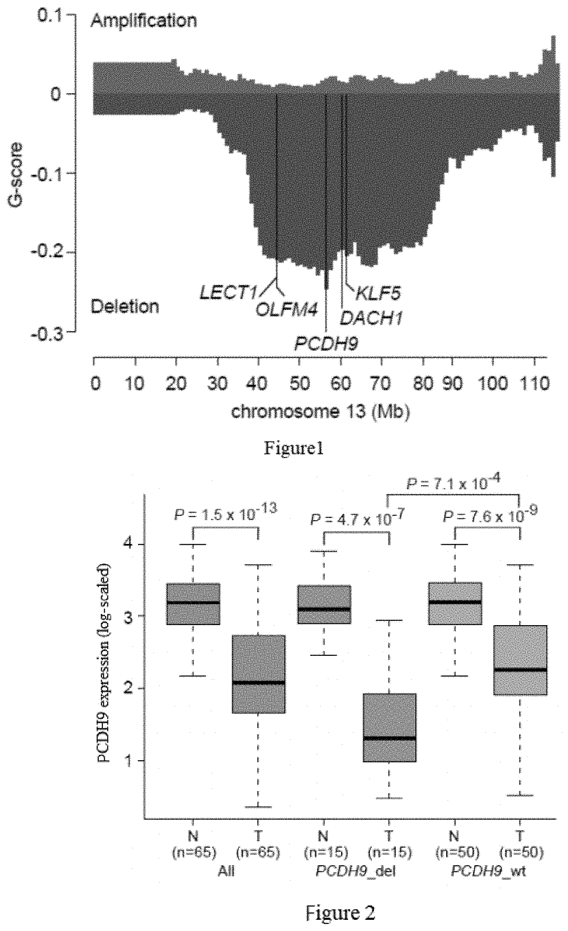

In the present invention sequencing was performed on 65 samples respectively for prostate cancer and adjacent tissues from the patients with prostate cancer to find that, deletion of the PCDH9 gene has occurred to chromosome 13 (as depicted by FIG. 1). In the same patient sample, significantly lower expression level of PCDH9 mRNA was seen in the prostate cancer tissues in comparison to the normal tissues, meaning the expression of PCDH9 mRNA is significantly decreased in the prostate cancer tissues compared to the normal adjacent tissues (FIG. 2). Hence it is suggested that the PCDH9 gene may be useful as a tumor suppressor gene in prostate cancer.

In the wild type of prostate cancer tissues with or without PCDH9 deletion, the expression level of PCDH9 is also significantly lower in the prostate cancer tissues in comparison to the corresponding normal tissues (FIG. 2), suggesting the correlation between the down-regulation in the expression of PCDH9 and the occurrence and advancement of prostate cancer.

(1.2) The close relationship between the decrease in the expression of PCDH9 mRNA in the prostate cancer tissues from the patients and deletion of the PCDH9 gene

As described above, it was shown there exists good relevance between the expression of PCDH9 mRNA and the evaluation of prostate cancer prognosis for the patient. The expression of PCDH9 mRNA in the patient can be related to multiple factors, among which chromosomal deletion could be one of the possible factors leading to the decrease in the expression of PCDH9 mRNA. The present invention disclosed there is a close relationship between the decrease in the expression of PCDH9 mRNA and deletion of the PCDH9 gene in patients with prostate cancer (FIGS. 1 and 2).

(1.3) The relationship between the down-regulation of the expression of the PCDH9 gene and the occurrence of metastatic prostate cancer

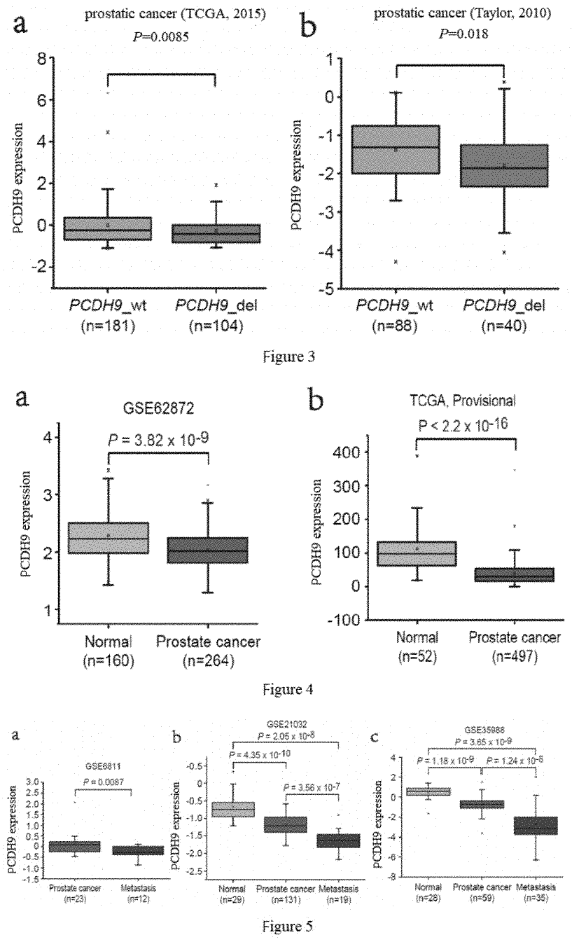

Upon examining the multiple independent sets of clinical statistics of the expression of PCDH9 mRNA, for example with the data from 2015 TCGA database (FIG. 3a) and the data from 2010 TCGA database (FIG. 3b), the present found that, a decrease in the expression was observed in the samples with deletion of PCDH9. Besides, analysis of the data from the GSE62872 database (FIG. 4a) and TCGA database (FIG. 4b) found the expression of PCDH9 mRNA in the prostate cancer tissues significantly decreases in comparison with that in the normal adjacent tissues.

Further, analysis of the data from the GSE6811 database (FIG. 5a), the GSE21032 database (FIG. 5b) and the GSE35988 database (FIG. 5c) found that, the expression of PCDH9 mRNA in the normal tissues from the patients with the prostate cancer is significantly higher than that in the tumor tissues (P_{GSE21032}=4.35×10⁻¹⁰, P_{GSE35988}=1.18×10⁻⁹), and even higher than that in the metastatic prostate cancer tissues (P_{GSE6811}=0.0087, P_{GSE21032}=2.05×10⁻⁸, P_{GSE35988}=3.65×10⁻⁹), suggesting the close relationship between the expression of PCDH9 mRNA and the degree of malignancy of prostate cancer, where the higher the degree of malignancy of prostate cancer, the lower the expression of PCDH9 mRNA goes.

(1.4) The relationship between the down-regulation of the expression of the PCDH9 gene and the high PSA content as well as the high clinical stage

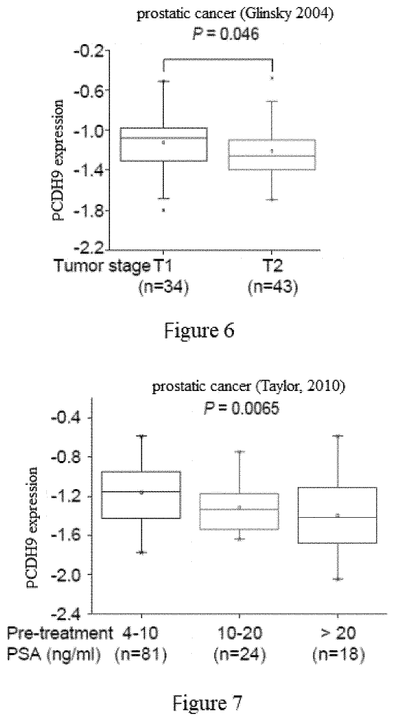

With the analysis of the difference in the expression of PCDH9 mRNA among prostate cancer tissues at various stages, the present invention found that, the expression of PCDH9 mRNA in the prostate cancer tissue at T2 stage is decreased compared with that at T1 stage (Ginsky, 2004, FIG. 6), suggesting the down-regulation of the expression of the PCDH9 gene is correlated with the high clinical stage of prostate cancer.

Prostate specific antigen (PSA) is a traditional diagnostic marker for prostate cancer, which is highly relevant with the degree of malignancy of prostate cancer. The analysis of data from Taylor 2010 (FIG. 7) found that, with the PSA level going up (4-10, 10-20, >20), the expression level of PCDH9 mRNA in the patients kept decreasing, suggesting the down-regulation of the expression of the PCDH9 mRNA gene is relevant with the high PSA level.

(1.5) The relationship between the decrease in the expression of PCDH9 mRNA and the time at which biochemical recurrence occurs

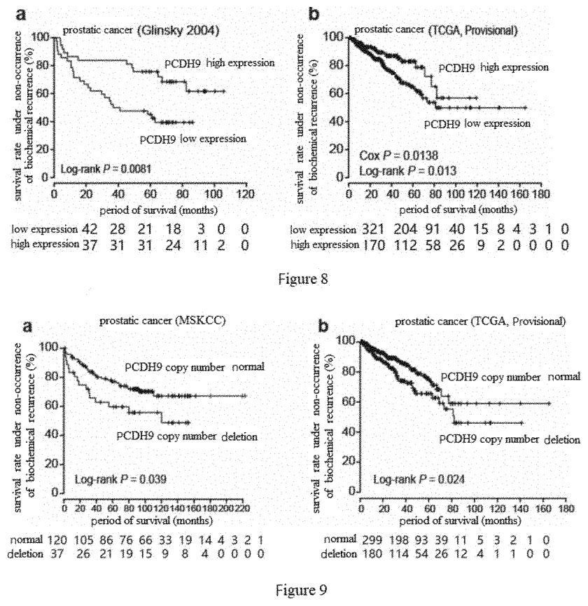

Bioinformatics was used in the present invention to analyze the expression of PCDH9 mRNA in the prostate cancer tissues from patients with prostate cancer, observing a significant difference in the evaluation of prostate cancer prognosis between patients with high expression of PCDH9 mRNA and those with low expression. The present invention applies the usual standard of evaluation of prostate cancer prognosis, where whether biochemical recurrence occurs is being referred to, as having been used in the prostate cancer prognosis researches(ie, two consecutive blood PSA levels after radical prostatectomy>0.2 ng/ml). After analyzing the present invention found that, in patients with decreased expression of PCDH9 mRNA observed were shortened period before biochemical recurrence actually occurred (the length of time is defined as the period since the operation of the radical surgery for prostate cancer till the occurrence of biochemical recurrence, and a shortened period of time suggested poor prognosis) (FIG. 8a and b).

(1.6) The relationship between deletion of PCDH9 gene and the occurrence of biochemical recurrence in patients with prostate cancer after radical surgery

As described above, the expression of PCDH9 mRNA is significantly correlated with the result of evaluation of prostate cancer prognosis, while the expression of PCDH9 mRNA is also closely related with deletion of the PCDH9 gene. In view of these, the present invention went on confirming whether there exists any relevance between deletion of the PCDH9 gene and the evaluation of prostate cancer prognosis.

The present invention found that, in patients with deletion of the PCDH9 gene, the occurrence of biochemical recurrence was much quicker to be observed in comparison with the case of their normal counterparts (FIG. 9a, b), meaning patients with deletion of the PCDH9 gene experience a significantly shortened biochemical recurrence time.

(1.7) The frequency of presence of deletion of the PCDH9 gene in metastatic prostate cancer

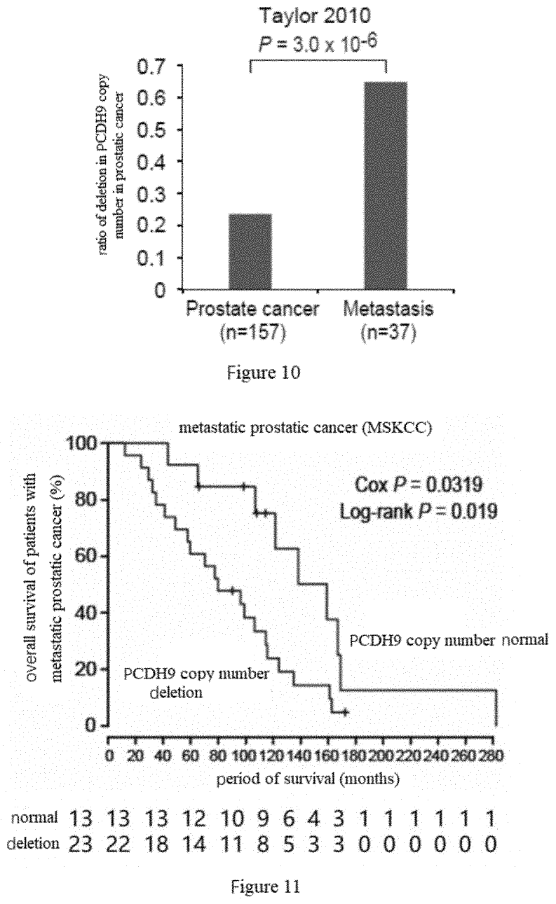

Compared with the population of western countries, Chinese patients have proved to include more cases where the cancer has already spread to other parts of the body at their initial diagnosis. It remains one difficult issue to perform evaluation of prostate cancer prognosis for patients with metastatic prostate cancer. Through analysis the present invention found that, the frequency of presence of deletion of the PCDH9 gene in patients with metastatic prostate cancer is significantly higher than the case of non-metastatic prostate cancer (FIG. 10).

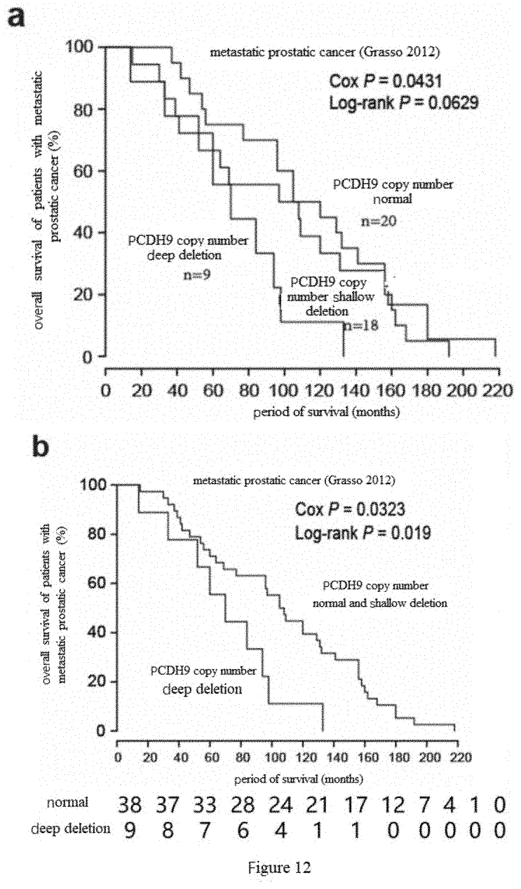

The present invention further analyzed the expression of PCDH9 DNA in the samples of patients with metastatic prostate cancer (FIG. 11 and FIG. 12a, b), finding the overall survival for patients with deletion of the PCDH9 gene is generally poorer than those without deletion.

In Summary, the researches in the present invention suggest that PCDH9 is useful as a tumor suppressor gene in the occurrence and advancement of prostate cancer, and that deletion of the PCDH9 gene as well as the decrease in the expression of PCDH9 mRNA provide important information for performing evaluation of prostate cancer prognosis.

### EXAMPLE 2

### The Significance Of PCDH9 In The Malignant Progression Of Prostate Cancer

(2.1) The relationship between the PCDH9 gene and the proliferation, metastasis and invasion of prostate cancer cells

None of the previous studies has touched upon the relationship between the PCDH9 gene and the malignant progression of prostate cancer.

As described above, the present invention has disclosed the close relevance underlying both the expression of PCDH9 mRNA and deletion of the PCDH9 gene with the evaluation of prostate cancer prognosis. As tumors are characterized as unlimited proliferation and invasion and metastasis, the present invention also studied the possible relevance underlying the PCDH9 gene and the proliferation, metastasis and invasion of prostate cancer cells.

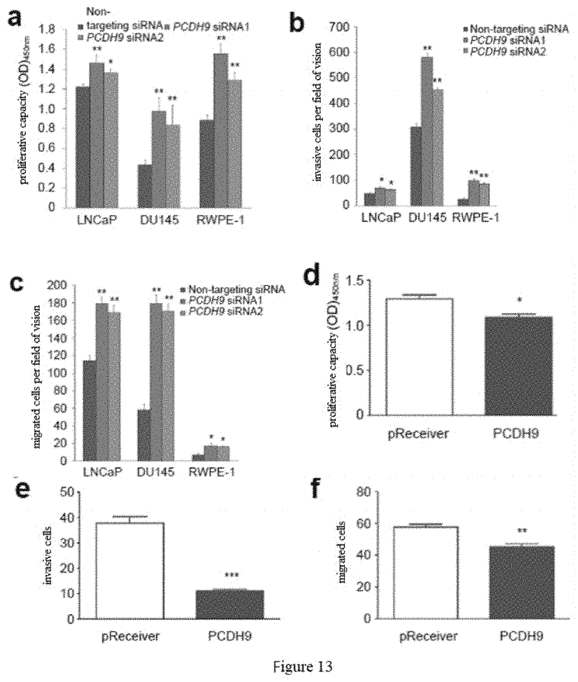

The present invention overexpressed the PCDH9 gene in the prostate cancer cell lines DU145 by means of lentivirus infection to determine the change in the proliferative capacity of cells by Cell Counting Kit-8 (CCK-8) assay (FIG. 13a, d). The change in the invasive (FIG. 13b, e) and metastatic (FIG. 13c, f) capacity of cells were determined by Transwell assay. The present invention found that after overexpression of the PCDH9, not only the proliferative capacity, also the migration and invasion ability of the tumor cells were diminished.

(2.2) Experiments with nude mouse models bearing subcutaneous tumors of prostate cancer cells

Nude mice were injected with prostate cancer cells DU145 with PCDH9 over expression (treatment group, DU145-PCDH9) and negative control group (DU145-pReceiver, in which pReceiver was an empty vector). The cells were allowed to grow and enter into the log phase when the cells are under the optimum status. Took 1×10⁶ cells from both the treatment and the control group, respectively mixed with Matrigel (Becton, Dickinson and Company) in the ratio of 1:1 by volume. Measured the size of the tumors of the mice every two days.

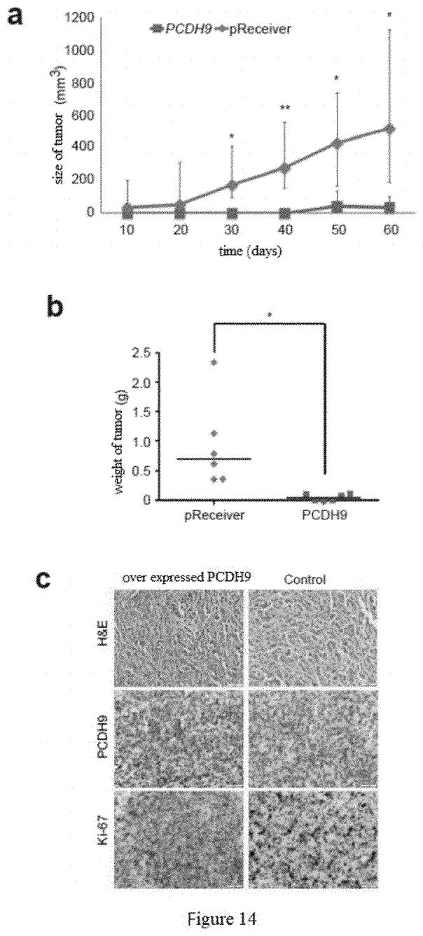

It was shown that the size and the weight of the tumor are significantly reduced in the nude mice that have been injected with prostate cancer cells DU145 with PCDH9 over expression (FIG. 14a, b).

According to the Immunohistochemistry (IHC)analysis, in the prostate cancer cells DU145 with PCDH9 over expression, the expression of PCDH9 was significantly up-regulated, suggesting a successful construction of the in vivo model, in which the exogenous expression of PCDH9 has been increased while the cell proliferation marker Ki-67 significantly down-regulated (FIG. 14c).

The above results indicated the over expression of PCDH9 diminished the proliferative capacity, migration and invasion ability of the prostate cancer cells.

(2.3) Genomic expression assay

In the present invention, genomic expression assay was conducted for both groups of DU145-PCDH9 cells and DU145-pReciever cells.

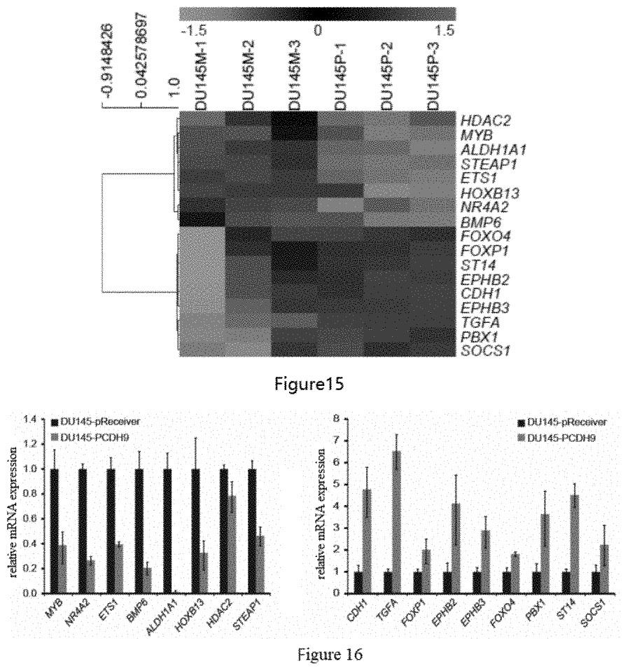

With gene expression profiling, the present invention found that, the expression of prostate cancer oncogenes (such as HOXB13 and ETS1) and of the marker of prostate cancer stem cells ALDH1A1 were down-regulated, the expression of transfer inhibitors (such as FOXOA and FOXP1) and epithelial-mesenchymal transition marker (CDH1) were up-regulated (FIG. 15).

Besides, the present invention verified the above changes in the expression of the relevant genes by real-time quantitative PCR, which produced similar results ,as shown in FIG. 16.

In summary, PCDH9 has shown a suppressing effect to the pathways in prostate cancer and is also useful in enhance the efficiency of the suppression pathway in prostate cancer to work as a tumor suppressor gene.

What is claimed:

1. A PCDH9 biomarker for molecular classification of prostate cancer and for evaluation of prostate cancer prognosis, wherein the PCDH9 biomarker includes a PCDH9 DNA, a PCDH9 mRNA or a protein encoded thereby; the mRNA sequence of PCDH9 is shown as SEQUENCE ID No. 1; the protein sequence encoded by the PCDH9 gene is shown as sequence ID No. 2.

2. Use of a reagent for detection of the PCDH9 biomarker according to claim 1, as a product for molecular classification of prostate cancer and for evaluation of prostate cancer prognosis, wherein the reagent for detection of the PCDH9 biomarker includes a PCDH9 DNA, an mRNA or a protein encoded thereby for specific detection of PCDH9.

3. An in vitro diagnostic product for prostate cancer, wherein the in vitro diagnostic product comprises a reagent for specific detection of PCDH9 DNA, and/or a reagent for specific detection of PCDH9 mRNA, and/or a reagent for specific detection of protein encoded by the PCDH9 gene.

4. The in vitro diagnostic product for prostate cancer according to claim 3, wherein the in vitro diagnostic product comprises a kit, a gene chip and a solid support; the solid support includes arrays, microarrays and protein arrays.

5. Use of the PCDH9 biomarker according to claim 1 in the preparation of pharmaceuticals for suppressing the proliferation, metastasis and invasion of prostate cancer cells.

6. Use of the PCDH9 biomarker according to claim 1 in the preparation of pharmaceuticals for suppressing the expression of prostate cancer oncogenes and for suppressing the expression of markers of prostate cancer stem cells.

7. The use according to claim 6, wherein the prostate cancer oncogenes include HOXB13 and ETS1; the marker of prostate cancer stem cells is ALDH1A1.

8. Use of the PCDH9 biomarker according to claim 1 in the preparation of pharmaceuticals for promoting the expression of metastasis inhibitors of prostate cancer cells and for promoting the expression of epithelial-mesenchymal transition markers.

9. The use according to claim 8, wherein the metastasis inhibitors of prostate cancer cells include FOXOA and FOXP1; the epithelial-mesenchymal transition marker is CDH1.

10. A method for molecular classification of prostate cancer and evaluation of prostate cancer prognosis in patients who are diagnosed with prostate cancer, comprising the following steps:
a) detecting the DNA copy number of the PCDH9 gene, or the expression levels of mRNA or the protein encoded thereby in pathology samples of prostate cancer;
b) dividing the patients into a low expression group and a regular expression group by referring to the result of the previous step, wherein the low expression group are comprised of such patients in whom are seen deletion or decrease in the DNA copy number of the PCDH9 gene, or decrease in the expression levels of mRNA or the protein encoded thereby, compared to their control counterparts, and who are given a bad prognosis indicative of deteriorating and highly invasive tumors, poor prognosis for survival, likelihood of biochemical recurrence, distant metastasis or disease progression or death; whereas the regular expression group is presented with a result of the previous step reflecting the opposite.


SEQUENCE LISTING (2 sequences)

SEQ ID NO: 1 - DNA, 6234 bp, Artificial Sequence
  Synthetic PCDH9 mRNA
agttcagagagaggagagggagaaggagagaggggcagaggggaagggagagagggagagcacgcgagacggaaaggagcgcctcagagtctctgaagcacgcaagagataaccgattaggaatttttcgggcaactgtcacccggatagctgtcagagaatcatcatcaccgcaactctgacgtttcctacaagaagttagagacttaagcagtattggcatcggatggaaatggtatgcatgctgctgtggaaaatatccctgagctgaagaagtgcaactatgtgttgtgtgtgctaaatgcccagaagaacccagacccagtgggtaagagagacgaaatgtggagagaatgactaacgacaaatccgtgaatttgttctctggagttgctaatttgccagcccgaagcaacacattttacaaggaggaaacattttgctgcttctgatttctccccaaactggtgctaccagatgcatggctttctatgtgttggaacaaatcattcctaactgcagcatactgggaaaggggaaaggttgaggcaactaaccggctgtctccaaataagtgatgagatataatgcatcctccagtccatgcacgcttcctcttgcaatttgtgcatcattcctcagtggaaacctttaaaccctaaaatccaggaaaagaaaataaatacattatcatggacctgagggatttttacctgttggctgctctgattgcctgtttaaggctggattccgcaatagctcaagaacttatttacactattagagaggaattgcctgaaaatgtgcccataggaaacataccaaaggatctgaacatttctcacatcaatgctgccacagggaccagcgccagccttgtctacagactggtttctaaagctggggatgcccctttggtgaaagtttccagcagcactggggaaattttcacaacctccaacagaatagacagagaaaaactctgtgctggcgcctcatatgctgaggagaatgagtgtttctttgaacttgaggtggtgatcctccccaatgatttcttcaggctgatcaaaataaaaataattgtcaaggataccaatgataatgcccccatgtttccatctcctgtcatcaatatttccattccagaaaacactttgatcaacagccgctttccaattccatcagcaacagatcctgacacaggcttcaatggtgtacagcattatgaattgttaaatgggcagagtgtttttggactggatatcgtggaaactccagagggagagaagtggccacaactgattgttcagcaaaacttggatagagaacagaaagatacctatgtgatgaaaatcaaagtagaggatggaggcactccacagaaatccagtacggccatactgcaggtcacagtaagtgatgtaaatgacaacaggccagtgtttaaagagggtcaagtggaggtgcatattccagagaatgctcccgtaggtacctctgtaattcagctccatgccactgatgcagatataggcagtaatgctgaaatccggtacatttttggtgcccaggtcgcccctgcaaccaaaagactctttgctttaaataatactactgggctgattacagttcagaggtccttagatagagaggagacagccattcacaaagtgacagtgctggctagtgacggcagctccactcctgctcgagcaacggttaccatcaatgtcaccgatgtaaatgataaccctcctaatatagacctcaggtacattataagtcccatcaatggcaccgtgtatttatctgagaaagatcctgtcaatacaaagattgccctaattacagtttcagataaggacacagatgtgaatggcaaagtgatctgttttattgaaagagaggtcccatttcatttgaaggcggtatatgacaaccaatatttgttagagacctcttctttgttggactatgagggcaccaaagaattcagctttaaaattgttgcctctgattctgggaagcccagtttaaatcagactgccctggtaagggttaagcttgaggatgaaaatgacaacccaccaattttcaaccagcctgtaattgagctgtcagtttctgaaaacaaccgacgtgggttatacttaacaactattagtgccacagatgaagacagtgggaaaaatgcagacattgtttatcagcttggaccgaatgcctccttctttgatctggaccgaaaaacaggagttttgacagcctccagagtatttgacagagaagaacaagaacgattcatttttacagtaactgccagggacaatgggacccctcccctccaaagccaagcggctgtgattgttactgttctggatgagaatgacaatagccccaagtttactcataatcattttcaattttttgtgtctgagaatctgccaaagtatagtactgtgggggtaatcacagtgacagatgcagatgctggagagaataaagctgtgactctttccattctaaatgacaatgataattttgtgttggatccctattctggagtcataaagtcaaatgtctcatttgatagagagcagcagagttcctacacttttgatgtcaaagccactgatggaggacaaccacctcgttcctctactgcaaaagtaactatcaacgtcatggatgtcaatgacaacagcccagttgtcatttctccaccgtctaatacttcctttaagttggtgcccctctcagccattcctggctccgtggtagcagaagtttttgcagtggatgttgacactggaatgaacgctgaactaaagtatactatagtgagtggaaacaataaaggcttattccggattgatccagtaacaggtaacattactctggaagaaaaaccagcacctactgatgtgggattgcatcgtttggtggtcaacataagtgacctggggtaccctaagtctttgcacacgcttgtgcttgtattcctttatgttaacgacactgctggaaatgcctcctatatctatgacttgatccgcaggactatggagaccccgttggacaggaacataggggatagtagccaaccctatcaaaatgaggactatctaaccatcatgattgccatcatcgccggtgccatggtggtcattgttgtgatcttcgtcaccgttctggtgcgctgtcgccatgcatcaaggttcaaagcagctcagaggagcaagcaaggtgccgaatggatgtccccaaaccaggagaacaagcaaaacaagaaaaagaaaagaaagaaaaggaagtctcccaaaagctctcttttgaactttgttactatcgaagagtccaaacccgatgatgcagttcatgaacctatcaatgggacaataagcctgccggctgaactggaggagcaaagtataggaagatttgactggggcccggcacctccaacaacattcaagcctaacagtcctgacctggccaagcactacaaatctgcttctccacagcctgcttttcatctcaaaccagacactccagtttccgtgaaaaagcaccacgtgattcaggaactccctttggacaacacctttgttgggggttgtgacaccctttctaaacgctcttccactagttcagatcacttcagtgcctcagagtgcagttcccaaggaggcttcaagacaaagggccccttacacaccagacagtgtaactcacacagcaaaagtgacaatattcctgtcactcctcagaaatgtcccagctccacgggtttccacattcaggagaatgaagaaagccattacgagtcgcagcgccgtgttacgtttcatctccctgatggctcccaggaaagctgcagtgacagtggtctaggagaccatgagccggtgggtagtggaaccctgatctcacaccctcttcctctggttcagccacaggacgaattctatgaccaggcctctccggacaagaggactgaagcagatggcaactctgatcccaactctgatgggcctttgggtccccgaggattagctgaagctacagagatgtgcactcaagagtgcttggttttgggtcactctgataattgctggatgcctcctggcttgggtccatatcaacaccccaaatctcctctctcaacctttgcaccccagaaagaatgggtgaagaaggacaagcttgtgaatgggcacaccctgaccagagcctggaaagaagacagcaacaggaaccagttcaatgaccgtaagcagtatggctccaatgaaggccatttcaacaatggcagccacatgacagacattcctctggcaaatctgaagtcttataagcaagcaggaggtgctactgagagtcctaaggagcaccaactctaagaaaaggctatatgggacctaataactttaaatagacatgctaataccgtgtgcgtcagagctttatgtattcaatagatctctacaactatgccccttctagcagggatacctataactttgtgttagcaccatggagaatacaatgtttttgcaacatatgagagaagatagttctagccatgcaattttgtttactagaattgattacactctccagagatctctccattgtgcaaattttttttattattgcattttattttgaccctggactgctgctatgaacaatagaatatgcatttggaaagtaagaaagtttcatggattagagtgactgaaaaacatatccagttagaaaattgtgcttttttttttttttttttttttttttgtcattgatttgtgctgggactgctatacttgacattatactgcaaacaaaacttaaagagataagcattaaacctattgtgctgttaacaacgttagtggacacttgtaagtcaatcagtgttaaagtatttgtaaatagaagcaagctattattaacaacttttgtgtacttataatgttcacctaaaaatgttgtagcataattctgtgttttatggttgcttctgtccttctttttatttacctctttgttgttatttttgttgtttctttcagtgaggtgtttctgtgttagtagtctgtcttgacacacattgttcaaacaatctcaagcacttgtgtttaaaaaaggttctgaaatcttgcttctcatatgatagcttatgatgccggaattctgtaaacaataaagatgaaagaaaaaatgaaaactttatacaaaaaaggacttctgcaaaagcttggaaattcagaacctattttttatcatcttattttcaaagtaggcaaaagcaaaatcacttaattagtaccattaagtgagagagtaatggaaaactgttatccatcaaaagtgatataatatcctgtacttgtcactcatgtaaaactaatgtatcaaaattagcatatatggaagaaaaatcactattcaaaaatagtttcaattgggcaaatgtgtgtctacaactttgttgaaatggcagagtctgtgttgatcctcatatacaataatgttgttgtaacacaagtgttattagaccatagccacaaaatatttaatgtgttgtgccttcatgatgtaacagaacattctccaggtagggtagttgggggaaataaagggataaatctctaattattgctgtttaactgtttgttatcatagttggtcaatgcctggcaggtaccagcatattgtcacttaggtcaaaccaataaagtgacaactaatgttaataagatctcagttgcacgtgcaaacaaatccaaattcgaacattcttaggaggtttttgttaaggcatgacagatgatttaaacaaataaatagcatgcaacaaaatgtctttattgaaagaagtggaagttatcttaatgccacggttcaccatcaatttaaaagtaaaaaaaacaaaaaaaaaacaaaaaatttaaaaaaaaggtttgctaatctgatagttaatttgttcttaccaaaaaataaaattactttgtaaatagcagttcacatttttccacagtataatggaaaataatgggtaggggtgcattgcttattgaagtacatttttgtcggtttgtgaggggtgtttgatgactttgacagaataaatatctaaacaattatccttgttactgctttgccagttctacgttatttacaattattcagctcttgcaataaatgttctgaattcctgaaaaaaaa

SEQ ID NO: 2 - protein, 1237 aa, Artificial Sequence
  Synthetic PCDH9 Protein
MDLRDFYLLAALIACLRLDSAIAQELIYTIREELPENVPIGNIPKDLNISHINAATGTSASLVYRLVSKAGDAPLVKVSSSTGEIFTTSNRIDREKLCAGASYAEENECFFELEVVILPNDFFRLIKIKIIVKDTNDNAPMFPSPVINISIPENTLINSRFPIPSATDPDTGFNGVQHYELLNGQSVFGLDIVETPEGEKWPQLIVQQNLDREQKDTYVMKIKVEDGGTPQKSSTAILQVTVSDVNDNRPVFKEGQVEVHIPENAPVGTSVIQLHATDADIGSNAEIRYIFGAQVAPATKRLFALNNTTGLITVQRSLDREETAIHKVTVLASDGSSTPARATVTINVTDVNDNPPNIDLRYIISPINGTVYLSEKDPVNTKIALITVSDKDTDVNGKVICFIEREVPFHLKAVYDNQYLLETSSLLDYEGTKEFSFKIVASDSGKPSLNQTALVRVKLEDENDNPPIFNQPVIELSVSENNRRGLYLTTISATDEDSGKNADIVYQLGPNASFFDLDRKTGVLTASRVFDREEQERFIFTVTARDNGTPPLQSQAAVIVTVLDENDNSPKFTHNHFQFFVSENLPKYSTVGVITVTDADAGENKAVTLSILNDNDNFVLDPYSGVIKSNVSFDREQQSSYTFDVKATDGGQPPRSSTAKVTINVMDVNDNSPVVISPPSNTSFKLVPLSAIPGSVVAEVFAVDVDTGMNAELKYTIVSGNNKGLFRIDPVTGNITLEEKPAPTDVGLHRLVVNISDLGYPKSLHTLVLVFLYVNDTAGNASYIYDLIRRTMETPLDRNIGDSSQPYQNEDYLTIMIAIIAGAMVVIVVIFVTVLVRCRHASRFKAAQRSKQGAEWMSPNQENKQNKKKKRKKRKSPKSSLLNFVTIEESKPDDAVHEPINGTISLPAELEEQSIGRFDWGPAPPTTFKPNSPDLAKHYKSASPQPAFHLKPDTPVSVKKHHVIQELPLDNTFVGGCDTLSKRSSTSSDHFSASECSSQGGFKTKGPLHTRQCNSHSKSDNIPVTPQKCPSSTGFHIQENEESHYESQRRVTFHLPDGSQESCSDSGLGDHEPVGSGTLISHPLPLVQPQDEFYDQASPDKRTEADGNSDPNSDGPLGPRGLAEATEMCTQECLVLGHSDNCWMPPGLGPYQHPKSPLSTFAPQKEWVKKDKLVNGHTLTRAWKEDSNRNQFNDRKQYGSNEGHFNNGSHMTDIPLANLKSYKQAGGATESPKEHQL